(19) United States
(12) Patent Application Publication
(10) Pub. No.: US 2015/0314088 A1
(43) Pub. Date: Nov. 5, 2015

(54) DEVICE FOR IMPROVING AIR FLOW THROUGH A NASAL CAVITY DURING PHYSICAL ACTIVITY SUCH AS SPORTING PURSUITS

(71) Applicants: Nicholas Jon EDE, Victoria (AU); Michael Ralph Burgess JOHNSON, Victoria (AU); Robert Hueston HUMANN, Victoria (AU)

(72) Inventors: Nicholas Jon EDE, Victoria (AU); Michael Ralph Burgess JOHNSON, Victoria (AU); Robert Hueston HUMANN, Victoria (AU)

(21) Appl. No.: 14/417,421

(22) PCT Filed: Jul. 27, 2012
(86) PCT No.: PCT/AU2012/000898

§ 371 Date: Jun. 1, 2015

(51) Int. Cl. A61M 15/08

(52) U.S. Cl. CPC A61M 15/085

(57) ABSTRACT

A device for improving flow of air through a nasal cavity of a wearer during activity such as cycling or running or like exercise substantially minimizing air flow resistance, the device including a substantially U-shaped body forming a bridge for spanning adjacent nasal cavities of a wearer; a first adjustable body and a second adjustable body mounted to opposite upright portions of the U-shaped body, the first and second bodies each having: a belt adjustable about an attachment mount on an upright portion of the U-shaped body; a rib extending outwardly and arcuately from a portion of the U-shaped body distal to the bridge, the each rib ending in a relatively large surface area portion connected to a portion of the belt, the large surface area portion of the each rib adapted for abutting internal wall surfaces of the nasal cavity of a wearer, and wherein the belt and rib form an open channel defining an air flow pathway within the nasal cavity; and whereby in an operating condition the belt or belts is tightened by a wearer to allow insertion, and thereafter loosened so as to urge the large surface area portion of the ribs against nasal cavity walls, whereby resistance to flow of air is substantially reduced, producing improved air flow capacity for sporting activities and the like.

### FIELD OF THE INVENTION

The present invention relates to an improved nasal cavity dilation device, which can be easily adjustable for insertion in and dilation of the nasal cavity of a person to enhance flow of air through the nasal passage. In particular, the present invention relates to an improved nasal cavity dilation device which is suitable for use during physical activities in which increased air flow capacity is required substantially without discomfort, and can be adapted for systemic drug delivery of a range of medicaments.

### BACKGROUND OF THE INVENTION

During sporting pursuits such as cycling, football, hockey and the like, it is common for an athlete to wear a resilient plaster applied over the bridge of the nose to externally expand the nostrils to assist opening the nasal passage so that an athlete can gain an advantage from improved air flow during sport play or exercise.

A problem associated with the foregoing approach is that the effect is limited by both the inherent resiliency of the plaster and its ability to exert a sufficient outward force to expand and maintain a nostril cavity in an open condition. In addition the plaster requires painful removal that could result in the tearing of skin.

Other devices are known for use in expanding the nasal cavity to try to improve air flow. One such device, disclosed in U.S. Pat. No. 3,710,799, describes a pair of cage structures joined together by a flexible chain of inter-locked links, the cages being slightly larger than the nostrils but insertable therein so that the nose holds the cages in place. This device confers a deal of discomfort for a wearer upon insertion because the device is generally larger than the corresponding orifice in which it is to inserted. Conversely, if the device is too small the device may be dislodged during physical activity. Such a device may be useful while a wearer is asleep, however is not practical for use during physical exercise because some parts of the cage structures provide a resistance to air flow therefore limiting air intake which is greater during physical activity.

Other devices are available that include a resilient plastic strip with widened ends. This type of device is usually bent prior to insertion with the wide ends being inserted into the nostrils. The extent of dilation of the nostril cavity thereafter depends on the resiliency of the plastic to return to its normal configuration, and the relative size of the nostrils. These type of dilators however are provided in one size hence can be very uncomfortable for some wearer's because the force generated by the resilient plastic often causes irritation to the inside lining of the nose, and for other wearer's the plastic strip may not provide effective dilation for improved air flow.

Other devices are disclosed in patent documents such as a device described in U.S. Pat. No. 5,895,409 that can be inserted within the nasal cavity. This device has a rigid structure and requires manufacturers to make different sized devices to cater for a range of cavity sizes. Also, problems frequently arise in a nasal application when a wearer exhibits a deviated septum. A deviated septum is a curvature in the septum, the cartilage and bone that separates the nostrils. A curved septum often renders one nasal cavity a different shape and size to its neighbour and inhibits airflow through one side of the nose and can result in airflow blockage through one nostril.

Prior art devices, of the type disclosed in U.S. Pat. No. 5,895,409 that are insertable within the nasal cavity, suffer the drawback that if one dilator of a symmetrical pair, to be inserted, is of a sufficiently small size to enter one nostril then the remaining dilator of the pair is too small to be effective in dilating the other nostril. Conversely, if one device of a symmetrical pair is sufficiently large to effectively dilate one nostril, its pair is often too large to be inserted in the other nostril.

There is thus an ongoing need for an improved device or means to enhance air flow through the nasal passage of an athlete during a sporting activity, which is comfortable, can be universally fitted, and effective in meeting the need for increased air intake during physical activity.

Notwithstanding the forgoing, breathing intake can be adversely affected by reason of viral infection. Viral infections including ‘the common cold’ can result in nasal and head congestion diminishing breathing performance. Often such ailments are not sufficient to wholly prevent a person from participating in a physical activity, however can be sufficient to detract from optimal performance. It would be advantageous to administer a medicament through the nasal passage while concurrently expanding the nasal passage so as to both improve intake of air and alleviate viral symptoms so that a person can participate in a sporting activity effectively even with a viral infection.

Devices are known which can be used to increase air flow through the nasal passage. For example, a range of contraptions are known that can be worn like a mask on the face of a sufferer to help maintain airways in an open condition. These types of devices involve complex designs that are very conspicuous on a wearer. Clearly such devices are not practical for use during sporting activities.

It should be understood that any reference to prior art does not constitute an admission of common general knowledge. The preceding discussion of the background art is intended to facilitate an understanding of the present invention only. It should be appreciated that the discussion is not an acknowledgement or admission that any of the matter referred to or documents identified was part of the common general knowledge in Australia, or any other country, as at the priority date of this application.

Throughout the specification the word “comprise” or variations such as “comprises” or “comprising” will be understood to imply the inclusion of a stated integer or group of integers but not to the exclusion of any other integer or group of integers.

It is an object of the present invention to provide a device, which addresses or at least ameliorates one or more of the difficulties of the prior art.

A preferred object of the invention is to provide a device which can be adjustable by a wearer to fit most nasal cavities as well as each nasal cavity independently and to urge dilation of the cavity to enable passage of air/fluid through the cavity, and is effective for improving air flow for greater intake during physical activity and delivery of medicament(s).

### SUMMARY OF THE INVENTION

In accordance with the invention there is provided a device for improving flow of air through a nasal cavity of a wearer during activity such as cycling or running or like exercise substantially minimizing air flow resistance, the device including:
a substantially U-shaped body forming a bridge for spanning adjacent nasal cavities of a wearer;
a first adjustable body and a second adjustable body mounted to opposite upright portions of the U-shaped body, the first and second bodies each having:
   a belt adjustable about an attachment mount on an upright portion of the U-shaped body;
   a rib extending outwardly and arcuately from a portion of the U-shaped body distal to the bridge, the each rib ending in a relatively large surface area portion connected to a portion of the belt, the large surface area portion of the each rib adapted for abutting internal wall surfaces of the nasal cavity of a wearer; and
   wherein the belt and rib form an open channel defining an air flow pathway within the nasal cavity; and
whereby in an operating condition the belt or belts is tightened by a wearer to allow insertion, and thereafter loosened so as to urge the large surface area portion of the ribs against nasal cavity walls, whereby resistance to flow of air is substantially reduced, producing improved air flow capacity for sporting activities and the like.

The present device is lightweight and provides less resistance to air flow than prior art devices. As a result, the instant device is useful for insertion during sporting activities such as cycling and the like which require greater intake of air, hence reduces the sense of obstruction felt when exhaling through the nose under duress.

Each adjustable body can be sized and positioned within adjacent nasal cavity to exert a positive pressure against the internal walls of the cavity thereby opening the cavity to enable passage of air and/or fluid therethrough and to retain the device in its desired position within the nostril.

In one embodiment the belt can include mating components on the rib and upright portion of the U-shaped body.

In an alternative embodiment, the belt can include:
a looped strap structure whereby a portion of the strap is integral with the large surface area portion of the rib, and whereby one end of the strap is fixedly mounted to an upright portion of the U-shaped body and the other end of the strap is adjustable relative to the fixed end;
a releaseable holding means mounted to an upright portion of the U-shaped body, whereby the holding means includes a fastening means which cooperates with the strap to allow the strap to tighten or loosen; and
wherein one end of the strap is adjustably received by the fastening means, and the looped structure retracted or expanded upon tightening and loosening respectively.

The strap can be adjusted by the holding means to provide a choice of one of a plurality of open structures. The wearer/user can select an appropriate size looped structure relative to the user's nasal cavity, and the fastening means can maintain the loop structure at a selected size in a holding condition. Hence the device can be adjusted in situ by a wearer to suit the wearers' specific air flow requirements.

The holding means is releasably lockable to maintain the loop structure and large surface area of the rib in a desired position. An advantage of the holding means is that the size of the looped structure can be maintained in an adjusted configuration to enable improved airflow that suits a wearers individual requirements.

The holding means can include a security tie member operatively connected thereto which is adapted for fine adjustment of the looped structure in situ. Hence the amount of adjustability of the device can be controlled by the wearer.

A substantial benefit in the reversible locking of the holding means is its use in changing the dimensions of the structure when the user has a different condition and the nasal cavity size has changed or where a user over-expands the device prior to insertion; the holding means allows for further fine adjustment. Such a condition can occur due to inflamed sinuses or because of colds, influenza and other nose affecting ailments or due to physical damage such as sunburnt noses, broken or damaged noses.

The holding means can comprise of mating or interlocking components on the upright portion of the U-shaped body and on one end of the strap to releasably lock the strap.

In one embodiment, the holding means can include a sleeve or housing having a series of internal teeth which resiliently and releasably engage with one end of the strap to reduce or enlarge the channel opening in the looped structure.

The device of the invention is suited to any size nostril, is economic, reusable and aesthetically pleasing. The device can be made from polymeric materials, and can deliver a range of agents including fragrance, natural products, essential oils, decongestants, medicated vapor such as Vicks^{R} and medicaments.

The device of the present invention can include a means for delivery of a medicament or medicated vapour. In one embodiment, the enlarged ends of the rib members can comprise a matrix of plastic material containing a saturated load of a drug for controlled delivery of the drug transdermally across the nasal cavity walls.

In a further embodiment, the enlarged surface area of the rib members can include a depot or reservoir for housing a medicament. The reservoir or depot can comprise a pitted surface applied to the enlarged surface area of the rib member, which in contact with the nasal mucosa releases an effective amount of an active agent. In storage, the enlarged surface area can be covered with a removable adhesive.

Alternatively, the large surface area portion of the ribs can include a series of spaced apart parallel grooves into which a medicament formulation can be deposited. When the large surface area portion of the ribs contacts internal nasal cavity walls, the grooves substantially minimize direct contact between the medicament formulation and nasal cavity wall surfaces. This is particularly useful where the medicated formulation is a medicated vapor.

In a further embodiment, the large surface area portion of the ribs can include a matrix suitable for release of volatile substances including amyl nitrite to treat heart disease such as angina and also to treat cyanide poisoning.

One form of matrix can be heterogeneous including a polymer throughout which there is dispersed a medicament, and wherein the matrix is partially sealed so as to define an air flow pathway such that incoming air flow initiates release of volatile agent within the polymer matrix.

The mode of delivery can include any of the following; (1) the “active agent” could be infused through the enlarged surface area portion of a rib using by infusing drug into a matrix (2) the “substance” could be deposited in dimples that contact the drug directly with the inside of the nose or (3) the “substance” could be placed in a patch (pad) which sticks to the enlarged area of a rib. Removal of a cover before insertion would expose the “substance” for transdermal delivery.

The enlarged surface area of a rib can include a pad designed to release a volatile substance over time. The pad can be a polymeric matrix including VICKS/essential oils/natural products/amyl nitrite. For extended release the pad might be various thicknesses. The pad can alternatively act as a depot system for release of an encapsulated solid, which solid can permeate through the walls of the depot at a predetermined rate depending on the thickness if the depot walls, and permeation characteristics of the solid and depot.

The advantage of the delivery of medicated vapour by the present device is that any substance such as‘Vapor Rub’can be held away from contact with the skin while allowing inhalation of medicated vapour. Hence the substance is able to function purely as a vapour for inhalation while substantially eliminating irritation that can otherwise be caused by direct contact with the skin. In one instance a medicated delivery system can be mounted on the body of the device.

In a further alternative of the present invention there is provided a nasal cavity dilation device for improving flow of air through a nasal cavity including:
an elongate U-shape body having an uppermost and lowermost portion, the lowermost portion forming a bridge being sufficiently wide to span a nasal septum, the device including a pair of symmetrical adjustable bodies mounted to the U-shape body distal to the bridge, the adjustable bodies including:
   a belt being fastened at one end to an upright portion of the U-shape body, the belt being adjustable by a releaseable locking means mounted on the U-shape body,; and
   a rib extending from an uppermost portion of the U-shape body downwardly and arcuately ending in an enlarged section adapted to abut against internal wall of a nasal cavity, whereby the enlarged section is connected to a portion of the belt; and
   wherein the belt forms a looped structure including the enlarged section of the rib so as to form an opening for ingress of air;
wherein the enlarged surface area of each rib comprises a matrix or depot comprising a medicament for release in contact with nasal cavity walls.

The rib member on each of the first and second bodies can include flattened sections or enlarged ends which terminate on a portion of the belt or strap, and abut against internal nasal cavity walls to improve a wearers level of comfort. The rib members can be moved laterally to expand or contract the looped opening channel formed by the strap structure by passing one end of the strap through the sleeve until a tooth in the sleeve engages an appropriate end section of the strap.

Further exemplifications of the present invention will be described as follows with reference to the accompanying drawings. A brief description of the drawings follows:

### BRIEF DESCRIPTION OF THE DRAWINGS

FIG. 1 is a schematic plan view from above of a device in accordance with a first embodiment of the invention.

FIG. 2 is a schematic front profile view of a body section of the embodiment in FIG. 1.

FIG. 3 is a schematic plan view of section A-A in the device of FIG. 1.

FIG. 4 is a schematic perspective view of the device in accordance with FIG. 1.

FIG. 5 is schematic side or end view of the device in accordance with FIG. 1.

FIG. 6 is a schematic front perspective view of a rib element according to a further embodiment of the invention.

FIG. 7 is schematic perspective profile of the rib element shown in FIG. 6.

FIG. 8 is an exploded schematic view of a device in accordance with the present invention without belt members showing various expanded/retracted rib positions.

### DETAILED DESCRIPTION OF THE PREFERRED EMBODIMENT WITH REFERENCE TO THE DRAWINGS

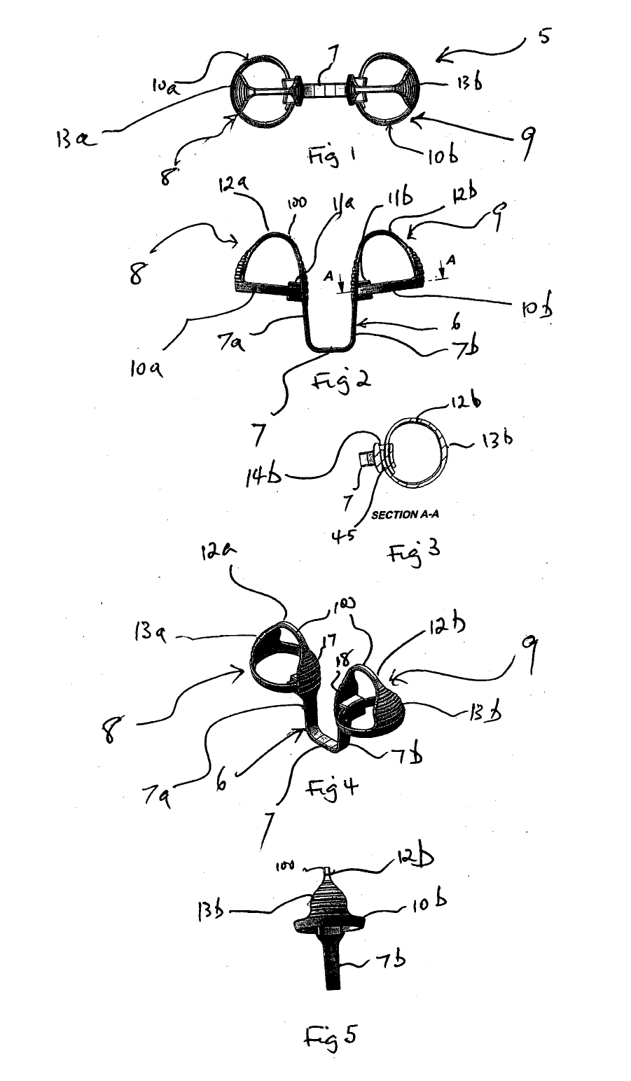

Referring to FIG. 1, there is shown an improved adjustable nasal cavity dilation device 5 insertable within a nasal cavity (not shown) for improving air flow during exercise or sporting activity such as cycling, football, hockey, basketball and the like.

The nasal cavity dilation device 5 includes a U-shape body 6 forming a bridge 7 separating adjacent upright support elements 7a and 7b, whereby the bridge 7 spans adjacent nasal cavities of a wearer (not shown) in an operating condition. As shown, the upright resilient support elements 7a and 7b of the U-shape body 6 diverge away from a central axis A-A′ (best seen in FIG. 8) to allow ready insertion.

The device 5 further includes a first 8 and second 9 adjustable bodies mounted to opposite upright portions 7a and 7b respectively of the U-shaped body.

The first 8 and second 9 body each include a belt 10a and 10b which describes a looped structure. The belts 10a and 10b are releasably adjustable about an attachment mount 11a and 11b on upright portions 7a and 7b of the U-shaped body. The bodies 8 and 9 also include a rib 12a and 12b extending outwardly and arcuately from an upper portion of the U-shaped body distal to the bridge.

As shown, each rib 12a and 12b includes a first narrow portion 100 dependent from the upper end portion of respective upright support elements 7a and 7b, and ending in a relatively enlarged surface area portion 13a and 13b which in turn is connected to a portion of the belt. The large surface area portion 13 a and 13b of each rib rests against internal wall surfaces of the nasal cavity of a wearer in an operating condition (not shown).

As shown, the each belt and respective rib form a substantially circular or elliptical opening through which air flows substantially free of obstruction.

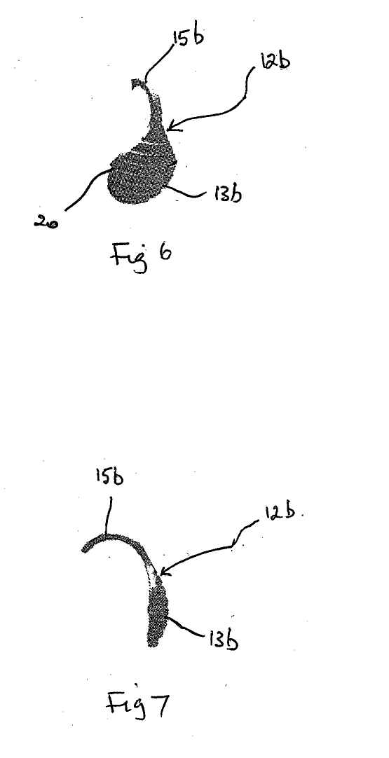

Referring to FIGS. 1 to 4, the attachment mount includes a releasable holding means 14a and 14b, which is mounted to a portion of the respective upright support 7a and 7b. The releasable holding means includes interlocking mating components on the upright support 7a and 7b, and strap portions of the belt. In the embodiment shown in FIG. 2 the holding means includes:
(i) a ratchet mechanism 14b (best seen in FIG. 3) mounted on a portion of upright support element 7b of the U-shape body, which is adapted to move between a holding and release condition, and
(ii) a ratchet strap 45 located on a portion of the belt 12b of the body 8.

As can be seen in FIG. 3, the strap is adapted to pass between the ratchet mechanism 14b and the upright support element 7b when the ratchet is in a release condition, and upon obtaining a desired holding condition the strap is held by the ratchet mechanism to fasten the belt about a portion of the upright support element in a holding condition.

In an operating condition the belt 10a and 10b of one or both bodies 8 and 9 is tightened by a wearer to allow insertion, and thereafter the belt is loosened by releasing the ratchet mechanism of the holding means so as to urge the large surface area portion of the ribs against nasal cavity walls.

Referring to FIG. 8, the enlarged surface area portion 13a and 13b of the ribs 12a and 12b, are biased towards an expanded condition (represented by X in FIG. 8).

For insertion of the device 5 within adjacent nasal cavities of a wearer, the belts 10a and 10b (not shown in FIG. 8) is/are tightened separately or together, to reduce the diameter of the looped structure and thereby urge the enlarged surface area portions of the ribs towards respective upright support elements. After insertion, the holding means is released to loosen the belt allowing lateral expansion of the enlarged surface area portion of the rib(s) away fro respective upright support elements.

As shown in the figures, the rib members 12a and 12b include a narrow portion 100 extending from an upper end portion of the upright support element(s), and an enlarged surface area portion 13a and 13b attached to a portion of the belt 10a/10b. The narrow portion 100 of the rib 12a acts as a living hinge allowing hinged movement of the enlarged surface area portion 13a when the respective belt is loosed or tightened. The narrow portions 100 of the ribs provides a substantial reduction in resistance to flow of air allowing use in sporting activities and the like which require increased requirement for respiration.

The device shown in FIGS. 1 and 7 can be retained in the nasal cavity in a suitably expanded state without inadvertent removal. The ribs can be maintained in a relative position by mating locking teeth in the belt and holding means. When the nostril cavity is expanded sufficiently the teeth can be interlocked by the releaseable holding means to retain the ribs in a new/expanded condition. When the enlarged surface area portions of a/the rib(s) are hingedly displaced between a constricted condition and an expanded condition when the belt is loosened, the enlarged surface area of each rib exerts a positive pressure against the internal walls of a nasal cavity.

In this embodiment the device can be adjusted manually insitu to open the nasal cavity. The upright support members of the U-shape body brace against one side of a nasal cavity. As best seen in FIGS. 1 to 5, internal surfaces of the opposite upright support elements 7a and 7b include a thickened region 17 and 18 to substantially reduce irritation with the nasal septum when inserted. The bridge 7 interconnecting the upright supports 7a and 7b prevents the device from being inadvertently inhaled by a user and acts as a rigid support for bracing a surface of the nasal cavity wall. When the device adopts an expanded condition the enlarged surface area portion of the ribs and the upright member (s) brace against the walls of the cavity to effectively prevent inadvertent removal of the device from the cavity.

The device shown in FIGS. 1 to 8 can be moulded in a single step process. The device 5 is symmetrical about axis A-A′, best seen in FIG. 8, hence for convenience an explanation has been provided for one half of the device which will in turn apply for the equivalent opposite symmetrical feature.

The enlarged surface area portion of the rib can comprise a flattened section or padded, which in an operating condition rests against internal wall surfaces of a nasal cavity. The flattening and broadening of the rib increases the surface area in contact with internal cavity walls to spread the concentration of force exerted by the device over a wider surface as practicable.

If it is desired to tighten the belt and thereby reduce the diameter of the looped structure adjustment is made by applying an external force against the enlarged surface area portion of the rib in the direction of X to Y as in FIG. 8. Conversely in order to apply an expanding force against the nasal wall, a user can exert a force against the enlarged surface portion in a direction of Y to X in FIG. 8. This will release the ratchet mechanism of the holding means and allow adjustment to an expanded looped structure.

In a further embodiment (not shown), the holding means can include mating teeth or a series of interlocking troughs and recesses. Once inserted in a nasal cavity a user can apply an outwardly directed force on the enlarged surface area portion of a rib. Once the rib is sufficiently laterally moved to promote increased airflow, the position of the enlarged surface area portion is releasably maintained by firm engagement of a trough between adjacent teeth within the recess.

In use the bridge 7 of the U-shape body is seated outside the nasal septum and upright members 7a and 7b include a raised or flat section 17, 18, which abuts internal wall structure of a nasal cavity. Generally at least the bridge portion of the body is transparent or flesh coloured so as to render the device inconspicuous from a casual observer, or fluorescent colored for persons who may want to display their use of the device or favorite colors.

In a further embodiment, the device of the present invention includes a means for delivery of a medicament or medicated vapour. In one embodiment (not shown), the enlarged ends of the rib members comprises a matrix of plastic material containing a saturated load of a drug for controlled delivery of the drug transdermally across the nasal cavity walls.

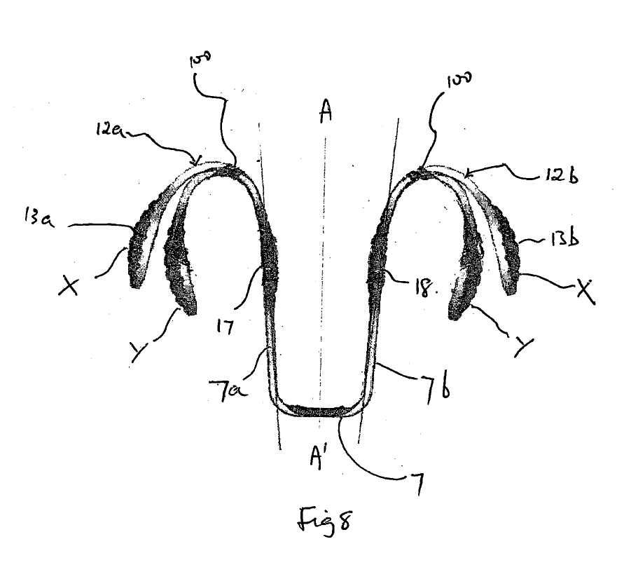

In a further embodiment best seen in FIG. 6, the enlarged surface area of the rib members includes a depot or reservoir for housing a medicament. The reservoir or depot comprise a grooved surface 20 applied to the enlarged surface area of the rib member. The enlarged surface as shown includes a series of spaced apart grooves into which a formulation of medicament or fragrance releasing agent or the like can be inserted. Once in the nasal cavity, the enlarged surface area portions of the ribs contact the nasal cavity walls substantially, without direct contact between the formulation and wall, whereby in contact with the nasal mucosa releases an effective amount of an active agent. In storage, the enlarged surface area can be covered with a removable adhesive.

The mode of delivery can include any of the following; (a) the “active agent” could be infused through the enlarged surface area portion of a rib using by infusing drug into a matrix (b) the “substance” could be deposited in dimples that contact the drug directly with the inside of the nose or (c) the “substance” could be placed in a patch (pad) which sticks to the enlarged area of a rib. Removal of a cover before insertion would expose the “substance” for transdermal delivery.

In a further embodiment (not shown), the enlarged surface area of a rib can be a pad designed to be a volatile substance that will release substance over time (like

VICKS/essential oils/natural products/amyl nitrite). For extended release the pad might be various thicknesses.

The advantage of the delivery of medicated vapour by the present device is that any substance such as‘Vapor Rub’can be held away from contact with the skin while allowing inhalation of medicated vapour. Hence the substance is able to function purely as a vapour for inhalation while substantially eliminating irritation that can otherwise be caused by direct contact with the skin. In one instance a medicated delivery system can be mounted on the body of the device.

The applicant does not intend to limit the invention to the disclosed embodiments, and any modifications or alterations that are obvious to a person skilled in the art from this disclosure are within the scope of this invention and covered herein.

Those of skill in the art will appreciate that any modifications or changes to the particular embodiments exemplified which can be made without that can be made without the need for inventiveness or departing from the spirit of the invention are included within the scope of the invention.

1. A device for improving flow of air through a nasal cavity of a wearer during physical activity such as sporting pursuits, the device including:
a substantially U-shaped body forming a bridge for spanning adjacent nasal cavities of a wearer;
a first adjustable body and a second adjustable body mounted to opposite upright portions of the U-shaped body, the first and second bodies each having:
a belt adjustable about an attachment mount on an upright portion of the U-shaped body;
a rib extending outwardly and arcuately from a portion of the U-shaped body distal to the bridge, the each rib ending in a relatively large surface area connected to a portion of the belt, the large surface area portion of the each rib adapted for abutting internal wall surfaces of the nasal cavity of a wearer; and
wherein the belt and rib form an open channel defining an air flow pathway within the nasal cavity substantially free of obstruction; and
whereby in an operating condition the belt or belts is tightened by a wearer to allow insertion, and thereafter loosened so as to urge the large surface area portion of the ribs against nasal cavity walls, whereby resistance to flow of air is substantially reduced producing improved air flow capacity for sporting activities and the like.

2. The device for improving flow of air through a nasal cavity of a wearer during physical activity such as sporting pursuits according to claim 1 wherein the belt includes:
a looped strap structure whereby a portion of the strap is integral with the large surface area portion of the rib, and whereby one end of the strap is fixedly mounted to an upright portion of the U-shaped body and the other end of the strap is adjustable relative to the fixed end;
a releaseable holding means mounted to an upright portion of the U-shaped body, whereby the holding means includes a fastening means which cooperates with the strap to allow the strap to tighten or loosen; and
wherein one end of the strap is adjustably received by the fastening means, and the looped structure retracted or expanded upon tightening and loosening respectively.

3. The device for improving flow of air through a nasal cavity of a wearer during physical activity such as sporting pursuits according to claim 2 wherein the holding means includes a security tie member operatively connected thereto which is adapted for fine adjustment of the looped structure in situ. Hence the amount of adjustability of the device can be controlled by the wearer.

4. The device for improving flow of air through a nasal cavity of a wearer during physical activity such as sporting pursuits according to claim 2 wherein the holding means includes mating or interlocking components on the upright portion of the U-shaped body and on one end of the strap to releasably lock the strap.

5. The device for improving flow of air through a nasal cavity of a wearer during physical activity such as sporting pursuits according to claim 2 wherein the holding means includes a sleeve or housing having a series of internal teeth which resiliently and releasably engage with one end of the strap to reduce or enlarge the channel opening in, the looped structure.

6. The device for improving flow of air through a nasal cavity of a wearer during physical activity such as sporting pursuits according to claim 1 further including a means for delivery of a medicament or medicated vapour.

7. The device for improving flow of air through a nasal cavity of a wearer during physical activity such as sporting pursuits according to claim 1 wherein the enlarged ends of the rib members include a matrix of plastic material containing a saturated load of a drug for controlled delivery of the drug transdermally across the nasal cavity walls.

8. The device for improving flow of air through a nasal cavity of a wearer during physical activity such as sporting pursuits according to claim 1 wherein the enlarged surface area of the rib members includes a depot or reservoir for housing a medicament.

9. The device for improving flow of air through a nasal cavity of a wearer during physical activity such as sporting pursuits according to claim 8 wherein the reservoir or depot can comprise a pitted surface applied to the enlarged surface area of the rib member, which in contact with the nasal mucosa releases an effective amount of an active agent. In storage, the enlarged surface area can be covered with a removable adhesive.

10. The device for improving flow of air through a nasal cavity of a wearer during physical activity such as sporting pursuits according to claim 8 wherein the large surface area portion of the ribs includes a series of spaced apart parallel grooves into which a medicament formulation can be deposited, whereby when the large surface area portion of the ribs contacts internal nasal cavity walls the grooves substantially minimize direct contact between the formulation and nasal wall surface.

11. The device for improving flow of air through a nasal cavity of a wearer during physical activity such as sporting pursuits according to claim 1 wherein the large surface area portion of the ribs includes a matrix suitable for release of volatile substances, amyl nitrite.

12. The device for improving flow of air through a nasal cavity of a wearer during physical activity such as sporting pursuits according to claim 11 wherein the matrix is heterogeneous including a polymer throughout which there is dispersed a medicament, and wherein the matrix is partially sealed so as to define an air flow pathway such that incoming air flow initiates release of volatile agent within the polymer matrix.

13. A nasal dilation device as substantially hereinbefore described with reference to the accompanying drawings.
